(19) United States
(12) Patent Application Publication
(10) Pub. No.: US 2026/0102545 A1
(43) Pub. Date: Apr. 16, 2026

(54) BREAST PUMP WITH SHABBAT MODE

(71) Applicant: Zev Supplies Corp., Pompano Beach, FL (US)

(72) Inventor: Dovid Rendler, Lauderhill, FL (US)

(21) Appl. No.: 19/355,596

(22) Filed: Oct. 10, 2025

Related U.S. Application Data
(60) Provisional application No. 63/706,008, filed on Oct. 10, 2024.

(51) Int. Cl. A61M 1/06

(52) U.S. Cl. CPC A61M 1/06; A61M 2205/276; A61M 2205/502; A61M 2205/583

(57) ABSTRACT

A breast pump system incorporating a Shabbat operational mode that enables use during periods when direct operation of electronic devices is prohibited. The system includes a microprocessor-controlled pump mechanism, a user interface with display and input controls, and firmware implementing multiple operational modes. The system allows a user to input desired parameters for the operation of the breast pump during the Shabbat period. Once the user selects the desired parameters and activates Shabbat mode, the pump operates autonomously according to the pre-programmed schedule whereby the pump becomes activated at selected intervals and duration periods. During this mode, all user input controls are disabled to prevent prohibited electronic interaction.

### RELATED APPLICATIONS

This application claims the benefit of pending U.S. Provisional Application Number 63/706,008, filed Oct. 10, 2024, the contents of which are incorporated by reference herein.

### FIELD OF THE INVENTION

The invention relates to the field of breast pumps, more particularly a breast pump having a mode configured to comply with Shabbat observance.

### BACKGROUND OF THE INVENTION

The Shabbat is a biblically pronounced “day of rest.” Adherents of the Shabbat and its laws are prohibited from engaging in certain work and work-related activities. Among the restricted work activities are operating electronic equipment. Nursing mothers who want to pump breast milk on Shabbat (or major Jewish holidays) are prohibited from powering electric pump on and off—which requires operation and manipulation of electronics and circuitry. There is therefore a need in the art for a breast pump having a control system that can be used on Shabbat and the holidays within the confines of Jewish law.

### SUMMARY OF THE INVENTION

Embodiments of the invention provide a system that includes a breast pump that is configured to run preset routines throughout a given time period so that a user may use the pump on Shabbat without having to engage with any electronic controls. In embodiments of the invention, the breast pump may be customized by a user prior to the onset of Shabbat to automatically run routines desired by the user.

### BRIEF DESCRIPTION OF THE FIGURES

FIG. 1 is a schematic view of a control panel for a breast pump in accordance with an embodiment of the invention.

### DETAILED DESCRIPTION OF THE INVENTION

Embodiments of the present invention will now be described with reference to the above-identified figures of the drawings. However, the drawings and the description herein of the invention are not intended to limit the scope of the invention. It will be understood that various modifications of the present description of the invention are possible without departing from the spirit of the invention. Also, features described herein may be omitted, additional features may be included, and/or features described herein may be combined in a manner different from the specific combinations recited herein, all without departing from the spirit of the invention.

In embodiments of the invention, a breast pump similar to those well-known in the art having electrical and mechanical components and a circuit board configured to control the breast pump is configured to include a Shabbat mode. The device is also provided with a user interface that may take the form of a touchscreen and/or physical buttons and which are used to control the device. The device may also include a separate display screen.

In embodiments of the invention, a breast pump in accordance with an embodiment of the invention is provided with software and/or firmware or combinations thereof that allow the unit to run a predetermined Shabbat routine (“Shabbat mode”). In accordance with embodiments of the invention, the breast pump includes at least a main control unit with a microprocessor and associated memory systems, a user interface with input and display capabilities, firmware for implementing multiple operational modes including a Shabbat mode, a real-time clock with battery backup, and digital storage media.

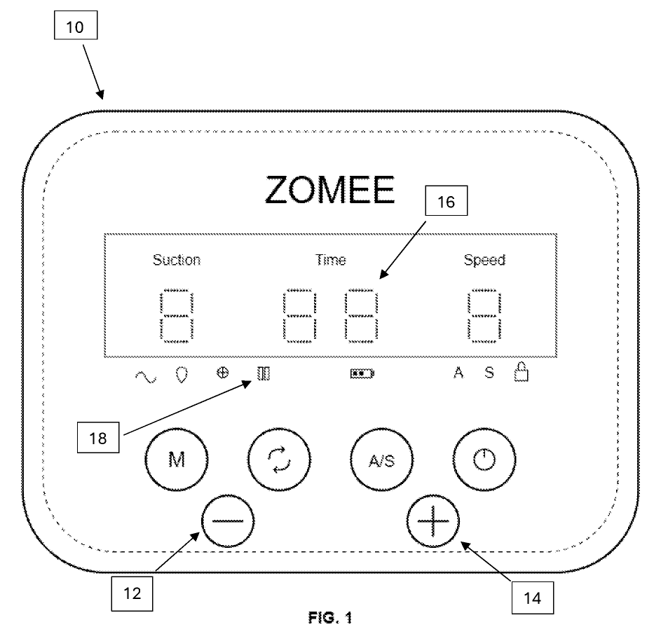

With reference to user interface 10 shown in FIG. 1, in an embodiment of the invention, prior to entering into Shabbot mode, the device is configured to switch into “Shabbat program mode.” In embodiment of the invention, Shabbat program mode is activated when a user activates a button or series of buttons. In Shabbat program mode, the system is configured to receive input from a user about desired pump settings. For example, in one configuration, when the device is switched to “off,” two different buttons are pressed in unison to activate Shabbat program mode. For example, in embodiments of the invention, minus button 12 and plus button 14 are pressed simultaneously to activate Shabbat program mode.

In embodiments of the invention, once switched to Shabbat program mode, the device prompts a user to enter desired pump settings.

For example, the device allows a user to select the frequency of when the pump is turned on, and for how long. For example, a user may select an “interval” of hours between each pumping session. For example, if a user selects an interval of four hours, then every four hours (or four hours from the end of a prior pumping), the pump is automatically turned on so that the user can pump as necessary without having to manually activate the unit to begin pumping.

With reference to FIG. 1, in embodiments of the invention plus button 14 is used to increase time, whereas the minus button 12 is pressed to decrease time. In embodiments of the invention, each press of the plus button adds an increment of time, such as for example, 15 minutes. Thus, pressing the plus button 14 four times will amount to an hour and so forth. This allows users to customize intervals. It will be understood that other increments are possible such as for example each press of the plus button equals 30 minutes. In other embodiments, each press provides 10 or even 5-minute increments allowing a user to fine tune the interval period. In embodiments of the invention, the “Time” section 16 of the display shows digital numbers responsive to each press of the plus and minus buttons 12, 14.

In embodiments, once the interval period is saved to the system, the system prompts a user to enter a “duration.” The duration controls how long the device continues to pump each time the pump is activated. In embodiments of the invention plus and minus buttons 12, 14 are configured to receive time input for the pump duration in a similar manner as described above. In embodiments, the time intervals may be five-minute intervals (i.e. each press adds five minutes), but other intervals are possible (e.g. 10, 20-minute intervals).

In embodiments of the invention, once the user inputs the interval and duration by, the user then pushes a button or series of buttons to indicate termination of Shabbat program mode and enter into Shabbat mode. In an embodiment of the invention, the same button or series of buttons that activates Shabbat program mode is configured to end Shabbat program mode and enter Shabbat mode when pressed a second time.

In embodiments of the invention, once Shabbat mode is activated, an indication (e.g. a pair of candles 18) is displayed on the device’s touchscreen which indicates that Shabbat mode is activated. In embodiments of the invention, in order to safeguard against the accidental activation of a control button during the course of Shabbat, in the Shabbat mode, all external buttons/switches/inputs become disengaged so that a user accidentally pressing a button will have no effect.

In embodiments of the invention, the pump pressure level or expression/stimulation setting will default to the setting that was most recently used by the user. In other embodiments of the invention, the during Shabbat program mode, the device prompts a user to input the suction level and/or speed.

In embodiments of the invention, in the Shabbat mode, the system is configured to display a countdown timer (e.g in time section 16) on the display screen to indicate how much time remains before the pump will activate next. In an embodiment, the time remaining is expressed in hours until there is one hour remaining, after which the clock switches to counting down in minutes. For example, if a user sets the interval to be four hours, then the display will show a four for the first hour of the interval, a three for the second hour, and a two for the third hour. In the final hour, the display will show a countdown from 60 minutes, thereby providing a user with a fine-tuned specificity as to when the device will begin pumping. In other embodiments of the invention, the countdown includes hours and minutes (until the final hour whereby only minutes are counted down).

In order to exit Shabbat mode and return the unit to normal (default) function, a user presses a button or series of buttons to exit Shabbat mode and return to default mode. In embodiments of the invention, the same button or buttons used to initiate Shabbat program mode and Shabbat mode are pressed a third time to terminate Shabbat mode.

It will be understood that the device requires a power source and, as such, the unit must be plugged into a wall during the course of Shabbat mode. In one embodiment, the Shabbat mode will only engage if the unit is connected to a power source. In another embodiment, Shabbat mode can be initiated in battery power mode (and the unit will cease operating once the battery in depleted).

In embodiments of the invention, the device is configured to automatically switch out of Shabbat mode and return to default mode after 73 hours (the maximum time for a consecutive Holiday and Shabbat).

In another embodiment of the invention, the breast pump system is configured to communicate with a mobile application installed on a user's mobile device such as a smartphone or tablet. The mobile application provides an alternative or supplementary interface for programming the Shabbat mode parameters, offering enhanced convenience and customization options for users.

In this embodiment, the breast pump includes wireless communication capabilities, such as Bluetooth or Wi-Fi connectivity, enabling bidirectional communication between the pump control unit and the mobile application. The application is configured with a dedicated Shabbat program mode interface that allows users to configure all operational parameters for the Shabbat mode.

The mobile application interface includes multiple input methods for user convenience. In one configuration, the application presents dropdown menus with preset interval options (e.g., every 2 hours, every 3 hours, every 4 hours, etc.) and duration options (e.g., 15 minutes, 20 minutes, 30 minutes, etc.) that users can select from. These preset options are based on common pumping schedules and can simplify the setup process for users.

Alternatively, the mobile application provides direct input fields where users can manually enter specific times for intervals and durations, offering greater flexibility and precision. For example, a user could input an interval of 3 hours and 45 minutes, or a duration of 23 minutes, which might be more difficult to achieve using physical button increments on the pump unit itself.

The mobile application further allows users to configure pump operation parameters including suction level, expression/stimulation patterns, and cycle speeds. These parameters can be set differently for each scheduled pumping session if desired. For instance, a user might prefer a specified suction level (e.g. stronger) for nighttime sessions and a different level (e.g. weaker) for daytime sessions.

In embodiments of the invention, the mobile application includes a scheduling preview feature that displays a timeline showing when the pump will activate throughout the Shabbat period based on the selected parameters. This visual representation helps users verify their settings and make adjustments before activating Shabbat mode.

The mobile application is configured to synchronize the programmed parameters with the breast pump's internal memory before Shabbat begins. Once synchronization is complete and Shabbat mode is activated, the wireless communication is disabled to comply with Shabbat observance, and the pump operates autonomously according to the pre-programmed schedule stored in its memory.

It will be understood that in embodiments of the invention, the app can run on the mobile device for the entirety of the Shabbat period to display countdowns to pumping initiation etc., but in alternative embodiments, the countdown is displayed on the device display panel. Still in other embodiments, the countdown is displayed on both the app and on the breast pump display panel.

In embodiments of the invention, the mobile application includes a profile storage feature allowing users to save multiple Shabbat mode configurations. This enables users to quickly select from previously saved settings for different occasions, such as regular Shabbat observance versus holiday periods which may require different pumping schedules.

Having described this invention with regard to specific embodiments, it is to be understood that the description is not meant as a limitation since further modifications and variations may be apparent or may suggest themselves to those skilled in the art. It is intended that the present application cover all such modifications and variations.

What is claimed:

1. A breast pump system comprising:
a pump mechanism configured to express breast milk;
a control unit comprising a microprocessor and memory;
a user interface comprising:
a display configured to show operational parameters; and
a plurality of input controls;
a real-time clock module; and
firmware executable by the microprocessor for alternatively implementing:
a normal operational mode wherein said pump mechanism is responsive to user activation of said input controls; and
a Shabbat mode wherein:
the pump mechanism operates autonomously according to pre-programmed parameters stored in said memory;
the input controls are disabled from affecting pump operation; and
wherein the pump mechanism automatically activates at predetermined time intervals without user intervention.

2. The breast pump system of claim 1, further comprising a Shabbat program mode wherein the system is configured to receive operational parameters prior to entering said Shabbat mode, whereby the operational parameters include at least an interval period between automatic pump activations and a duration period for each pump activation.

3. The breast pump system of claim 2, wherein entry into said Shabbat program mode requires simultaneous activation of at least two of the input controls.

4. The breast pump system of claim 3, wherein the at least two input controls comprise a plus button and a minus button.

5. The breast pump system of claim 2, wherein the interval period is adjustable in increments between 5 minutes and 60 minutes and the duration period is adjustable in increments between 1 minute and 30 minutes.

6. The breast pump system of claim 2, wherein transition from said Shabbat program mode to said Shabbat mode occurs upon a second activation of the same control sequence used to enter said Shabbat program mode.

7. The breast pump system of claim 1, wherein said display shows a visual indicator when said Shabbat mode is active.

8. The breast pump system of claim 7, wherein said visual indicator comprises a representation of one or more candles.

9. The breast pump system of claim 1, wherein during said Shabbat mode, the display shows a countdown timer indicating time remaining until the next automatic pump activation.

10. The breast pump system of claim 9, wherein said countdown timer displays time in hour increments when more than one hour remains and displays time in minute increments when one hour or less remains.

11. The breast pump system of claim 1, wherein the Shabbat mode automatically terminates after a predetermined maximum duration.

12. The breast pump system of claim 11, wherein the predetermined maximum duration is 73 hours.
